(19) United States
(12) Patent Application Publication
(10) Pub. No.: US 2025/0380887 A1
(43) Pub. Date: Dec. 18, 2025

(54) Image Processing and Reflection-Absorption Spectroscopic Method and Apparatus for Detection of Carotenoids in Human Tissue for Health Monitoring

(71) Applicant: Mohsen Sharifzadeh, Cottonwood Heights, UT (US)

(72) Inventor: Mohsen Sharifzadeh, Cottonwood Heights, UT (US)

(21) Appl. No.: 18/747,397

(22) Filed: Jun. 18, 2024

(51) Int. Cl. A61B 5/145; A61B 5/103; A61B 5/1455; G06T 7/90; H04N 9/64

(52) U.S. Cl. CPC A61B 5/14546; A61B 5/1032; A61B 5/1455; G06T 7/90; H04N 9/64; A61B 2560/0223; G06T 2207/10024; G06T 2207/30088

(57) ABSTRACT

This invention introduces a non-invasive method and device for detecting and quantifying antioxidant carotenoids in human tissues, such as skin, gum, and retina, using CIE Lab* color space analysis. The method employs absorption spectrophotometry and RGB image processing, providing a multifaceted approach to health monitoring. It detects specific antioxidants like lycopene, beta-carotene, lutein, and zeaxanthin. The system uses three methods: (1) pure absorption spectroscopy for precise antioxidant measurement; (2) an RGB camera technique with advanced image processing for diffuse spectra analysis; and (3) a hybrid model combining multiple spectroscopic technologies. A mobile application facilitates real-time health monitoring. Validated against Raman spectroscopy, the system ensures rapid, accurate, and reliable measurements without invasive sampling. Applications range from routine check-ups to targeted nutritional assessments, making it valuable for healthcare providers, researchers, and individuals.

### FIELD OF INVENTION

This invention relates to a non-invasive method and device for detecting and quantifying carotenoid antioxidants in human tissues, specifically utilizing the CIE Lab* color space for image processing. The method provides a versatile approach to health monitoring, with applications in skin, gum, and retina analysis.

### BACKGROUND OF THE INVENTION

Carotenoids, such as lycopene, beta-carotene, lutein, and zeaxanthin, are critical antioxidants that play a significant role in maintaining overall health. These compounds are known to protect cells from oxidative damage, support immune function, and prevent various diseases. Despite the recognized importance of carotenoids, there is currently a lack of non-invasive methods for accurately measuring their levels in human tissues.

Traditionally, methods like Raman spectroscopy and spectrograph-based diffuse reflection spectroscopy have been used to measure carotenoid levels. However, these techniques often require expensive equipment and are not easily accessible for routine health monitoring. The need for a cost-effective, user-friendly, and non-invasive method to measure carotenoids in human tissues is evident.

This invention introduces a novel approach that uses image processing based on the CIE Lab* color space to provide accurate, real-time measurements of carotenoids. By leveraging existing technologies, such as mobile cameras, and developing new algorithms for image analysis, this invention aims to make health monitoring more accessible and convenient.

The ability to measure carotenoid levels in various tissues, such as skin, gum, and retina, opens new possibilities for health monitoring. For instance, assessing carotenoid levels in the gum can provide insights into oral health, while measuring them in the skin and retina can indicate overall antioxidant status. This dual application enhances the utility of the invention, making it a versatile tool for preventive health care and personalized nutrition.

The invention also includes a method to account for skin pigmentation variations by normalizing the measurements based on the lightness (L*) value from the CIE Lab* color space. This ensures that the measurements are accurate regardless of skin tone. Additionally, a mobile application facilitates health monitoring by allowing users to take photos of their skin, gum, or retina. This feature makes the technology accessible to a broader audience and promotes proactive health management.

### DETAILED DESCRIPTION OF THE INVENTION

### 1. Methodology

Image Processing Technique: The image processing technique employs RGB cameras to capture images of the tissue, which are then analyzed using the CIE Lab* color space. This method provides a non-invasive way to quantify carotenoid levels based on their color characteristics.

### 1. Capture RGB Image:

Take an RGB image of the tissue (e.g., skin, gum, or retina) using a standard camera or mobile phone camera with a white LED light source.
Calibration Step: Prior to measurement, capture an image of a standardized calibration card with known color values to adjust for any variations in the camera and lighting.

### 2. Convert to CIE Lab Color Space:

Convert the RGB image to the CIE Lab* color space using standard conversion algorithms. These algorithms transform the RGB values to the L*, a*, and b* components, which represent lightness, the green-red axis, and the blue-yellow axis, respectively.
Convert the RGB image to the CIE Lab* color space, which separates the image into three components:
   L* (Lightness): Represents the lightness of the color, ranging from 0 (black) to 100 (white).
   a* (Green-Red Axis): Represents the color range from green (−) to red (+).
   b* (Blue-Yellow Axis): Represents the color range from blue (−) to yellow (+).

### 3. Analyze Color Components:

Measure the yellowness (b*) as an indicator of carotenoids, which are yellow. The redness (a*) and lightness (L*) components are also analyzed to provide a comprehensive assessment of the tissue's color characteristics.

### 4. Skin Pigmentation Correction:

Adjust the b* value based on the L* value to account for variations in skin pigmentation. This correction ensures that the measurements are accurate regardless of skin tone.
Example Correction Formula:

$$Adjusted b *= b * \times ( 1 + \frac{100 - L *}{100} \times k )$$

Where k is a scaling factor determined experimentally.

### 5. Calibration for Device Variability:

Calibration Card/Paper: Use a standardized calibration card to capture a reference image. The app adjusts the camera settings based on this reference to account for variations in the white LED and RGB sensors across different devices.
Device-Specific Calibration: Include a database of device-specific calibration settings. Users select their device model, and the app applies the appropriate calibration adjustments.
Dynamic Calibration Algorithm: Implement an algorithm that dynamically adjusts the captured image based on its properties, such as color balance and exposure, to ensure consistency across different devices.

### 6. Calculate Carotenoid Levels:

Use the adjusted b* value to quantify the level of carotenoids in the tissue. Higher adjusted b* values correlate with higher carotenoid levels.

### 2. Technical Details

### Light Sources:

Utilize a white LED light source to illuminate the tissue during image capture. The light source should be consistent to ensure accurate and repeatable measurements.

### Data Processing:

Process the captured images using algorithms that convert RGB to CIE Lab* and analyze the color components. The algorithms account for variations in lighting, tissue properties, and skin pigmentation to ensure accurate measurements.

### 3. Potential Applications

### Health Monitoring:

The device can be used to monitor carotenoid levels in various tissues, such as skin, gum, and retina, providing insights into overall health and aiding in the prevention and management of diseases.

### Mobile Health Applications:

An accompanying mobile application allows users to easily monitor their health status by taking photos of their skin, gum, or retina, making health monitoring accessible and user-friendly.
The app includes calibration features to ensure consistent and accurate measurements across different mobile devices.

### BRIEF DESCRIPTION OF THE DRAWINGS

FIG. 1: Tissue Absorption Spectrum

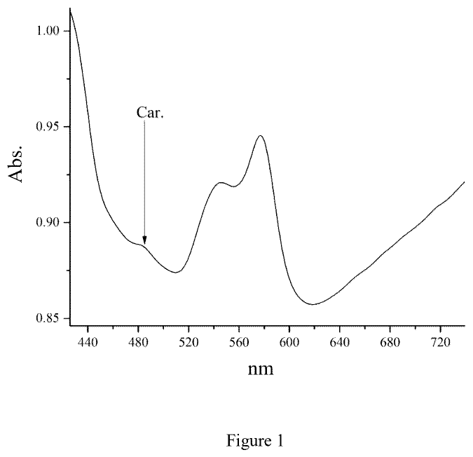

Illustrates the absorption spectrum of human tissue, highlighting the specific wavelength range where carotenoids are absorbed.

FIG. 2: Detection of 632 nm HeNe Laser Light

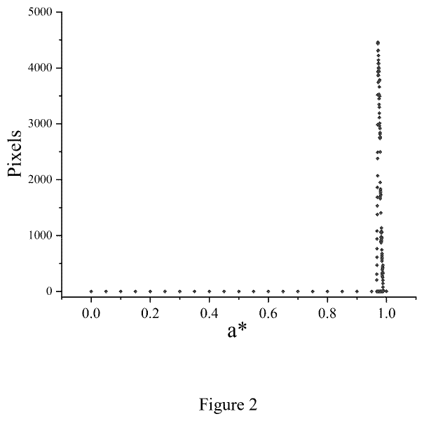

Shows the pixel intensity plotted against the a* value in the CIE Lab* color space for detecting the presence of a 632 nm HeNe laser light on a white target.

FIG. 3: Carotenoid Detection in Human Tissue Samples

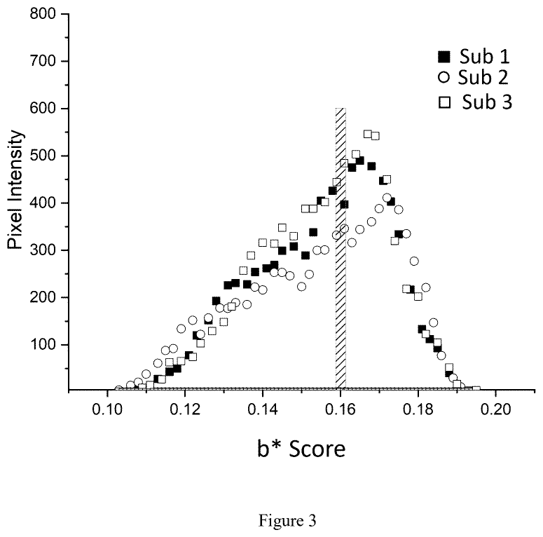

Displays the pixel intensity distribution at different b* values (yellowness) for three different subjects, indicating variations in carotenoid levels among them.

FIG. 4: Comparison of Carotenoid Scores

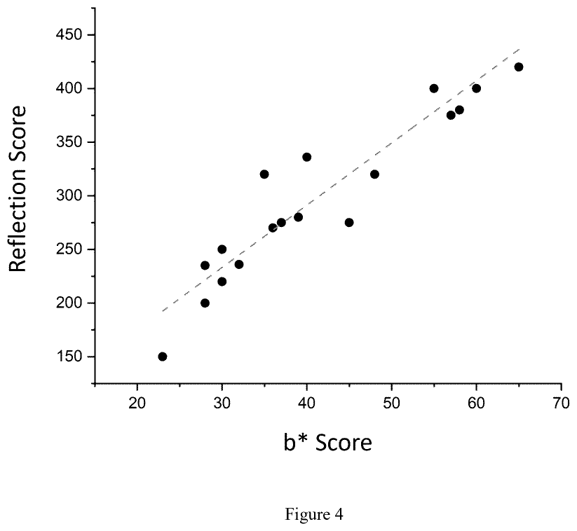

Depicts the correlation between carotenoid scores measured using diffuse reflection and those derived from the b* value for a group of subjects.

1. A method for non-invasive quantification of carotenoids in human tissue. Transitional Phrase: Comprising: Claim Body:
Capturing an RGB image of human tissue, such as skin, gum, or retina, using a device equipped with an RGB camera and white LED light source;
Converting the RGB image to the CIE Lab* color space using standard conversion algorithms;
Measuring the b* value in the CIE Lab* color space as an indicator of carotenoid levels;
Adjusting the measured b* value based on the L* value to account for variations in skin pigmentation;
Calculating the carotenoid concentration in the tissue based on the adjusted b* value.

2. The method of claim 1, Incorporates by Reference: Wherein the adjustment of the b* value based on the L* value is performed using a correction algorithm that accounts for the lightness (L*) of the tissue.

3. The method of claim 1, Incorporates by Reference: Wherein the device includes a standardized calibration card that is used to calibrate the RGB camera before capturing the image of the tissue.

4. The method of claim 1, Incorporates by Reference: Wherein the carotenoid concentration is calculated using a model that correlates the adjusted b* value with known carotenoid concentrations obtained from reference samples.

5. Preamble: A device for non-invasive measurement of carotenoids in human tissue. Transitional Phrase: Comprising: Claim Body:
An RGB camera configured to capture images of human tissue;
A white LED light source integrated with the device to provide consistent illumination during image capture;
Image processing algorithms configured to convert captured RGB images to the CIE Lab* color space;
A correction module that adjusts the b* value based on the L* value for skin pigmentation variation;
A computation module that calculates carotenoid levels based on the adjusted b* value.

6. The device of claim 5, Incorporates by Reference: Wherein the device includes a mobile application that allows users to capture images, process the images using the CIE Lab* color space, and display the calculated carotenoid levels.

7. The device of claim 5, Incorporates by Reference: Wherein the device is capable of detecting specific carotenoids, including lycopene, beta-carotene, lutein, and zeaxanthin.
